(19) United States
(12) Patent Application Publication
(10) Pub. No.: US 2025/0195214 A1
(43) Pub. Date: Jun. 19, 2025

(54) HEART VALVE PROSTHESIS AND METHOD FOR MANUFACTURING A HEART VALVE PROSTHESIS

(71) Applicant: NVT AG, Morges (CH)

(72) Inventors: Maximilian Kuetting, Gaertringen (DE); Steffen Westermann, Herrenberg (DE); Fenja Alkemper, Tuebingen (DE)

(21) Appl. No.: 18/962,916

(22) Filed: Nov. 27, 2024

Related U.S. Application Data
(63) Continuation of application No. PCT/EP2023/064079, filed on May 25, 2023.

(30) Foreign Application Priority Data
May 30, 2022 (DE) 102022113584.5

(51) Int. Cl. A61F 2/24; A61F 2/00; A61F 2/90

(52) U.S. Cl. CPC A61F 2/2418; A61F 2/2415; A61F 2002/0081; A61F 2/90; A61F 2210/0076; A61F 2230/0004; A61F 2230/0017

(57) ABSTRACT

The present invention concerns a heart valve prosthesis for implantation into the heart of a patient, wherein the heart valve prosthesis comprises a stent frame and a plurality of cells arranged in circumferential rows, a valve element being mounted to the prosthetic heart valve and comprising at least two, preferably three, leaflets, a skirt attached to the stent frame, wherein the skirt comprises a skirt main body and at least two, preferably three, skirt flaps being integral with or attached to the skirt main body, wherein the skirt main body covers at least the proximalmost row of cells of the stent frame, wherein each of the skirt flaps, in a folded state, forms a decoupling element, and wherein each of the skirt flaps covers one commissure cell of the distalmost row of cells of the stent frame, and wherein the leaflets, via a decoupling element, are, respectively, mounted to the prosthetic heart valve within the commissure cells of the distalmost row of cells.

### CROSS REFERENCES TO RELATED APPLICATIONS

This application is a continuation of international patent application PCT/EP2023/064079, filed on May 25, 2023, designating the U.S., which international patent application has been published in English language and claims priority from German patent application DE 10 2022 113 584.5, filed on May 30, 2022. The entire contents of these priority applications are incorporated herein by reference.

### FIELD OF THE INVENTION

The present invention relates to a heart valve prosthesis for implantation into a body lumen, in particular via transluminal delivery. The heart valve prostheses presented herein are particularly suited for use in replacing a native heart valve within a patient, as well as to a method for making or manufacturing a heart valve prosthesis.

### BACKGROUND

Heart valve diseases continue to be a significant cause of morbidity and mortality, and the replacement of diseased native heart valves with heart valve prostheses has become a routine surgical procedure for patients suffering from valve regurgitation or stenotic calcification of the leaflets.

Heart valve replacement is necessary where the native heart valve is damaged, mal- or nonfunctioning. In the heart, cardiac valves maintain the unidirectional flow of blood by opening and closing depending on the difference in pressure on each side. The mammalian heart comprises four chambers, i.e. two atria, which are the filling chambers, and two ventricles, which are the pumping chambers. In a mammalian heart, there are four heart valves present which normally allow blood to flow in only one direction through the heart, whereby a heart valve opens or closes depending on the differential blood pressure on each side.

The four main valves in the heart are the mitral valve, representing a bicuspid valve, and the tricuspid valve, which are between the upper atria and the lower ventricles, respectively, and thus are called atrioventricular (AV) valves. Further, there are the aortic valve and the pulmonary valve which are in the arteries leaving the heart. The mitral valve and the aortic valve are in the left heart and the tricuspid valve and the pulmonary valve are in the right heart.

The valves incorporate leaflets or cusps, wherein each valve has three cusps, except for the mitral valve, which only has two. E.g., the aortic valve is composed of 3 leaflets, wherein the leaflet cusps are named Right Coronary Cusp (RCC), Left Coronary Cusp (LCC) and Non-Coronary Cusp (NCC). Each cusp has two commissures, which are shared with the neighboring cusp. A commissure is the space or area where two leaflets abut and merge with the aortic wall. They act as support to the base structure of the cusps.

Ideally, native leaflets of a heart valve move apart from each other when the valve is in an open position, and meet or “coapt” when the valve is in a closed position. As such, a heart valve can be affected by a range of diseases and can, therefore, require cardiac valve replacement. The valve can either become leaky, i.e. regurgitant or insufficient, in which case the aortic valve is incompetent and blood flows passively back to the heart in the wrong direction. Further, the valve can become partially shut, i.e. stenotic, in which case the valve fails to open fully, thereby obstructing blood flow out from the heart. The two conditions frequently co-exist.

Until recently, the vast majority of heart valve replacements required full sternotomy and placing the patient on cardiopulmonary bypass. Traditional open surgery inflicts significant patient trauma and discomfort, requires extensive recuperation times and may result in life-threatening complications. To address these concerns, within the last twenty years, efforts have been made to perform cardiac valve replacements using minimally-invasive techniques, such as a percutaneous entry with a transluminal delivery. These percutaneous heart valve replacement therapies use a catheter to deliver a valve prosthesis to diseased site using a patients' lumen of the vascular system.

In general, two types of prosthetic heart valve devices are used in the industry to replace defective native heart valves, i.e. mechanical prosthetic valve devices and biological prosthetic valve devices. Biological prosthetic valve devices use a natural tissue, typically of mammalian, e.g. porcine or bovine origin, to form the collapsible leaflets of the biological prosthetic valve device.

While great efforts have been put into developing heart valve prostheses, existing valve prostheses suffer from a number of drawbacks, including premature failure due to wear, complexity of manufacture, and less than optimal performance, which deficiencies are present both in the valve component and the frame of existing heart valve prostheses. For example, a less than optimal leaflet design can result in inferior sealing of the fluid passageway and/or in undesirable overlap and/or crimping of the collapsible leaflets during a closure state. Also, the frames, which can act as stent components when inserted, often lack an adequate structural support for commissures and/or of suitable geometry for properly anchoring a valve component. A major drawback of the majority of percutaneous heart valve prostheses is their lack of a flexible attachment of the leaflets to the support structure, i.e. stent frame. Surgically implanted prostheses have been developed to incorporate flexible sections in the regions of the commissural attachment points. This feature has been shown to improve the overall durability of the prostheses by reducing the level of stress that remains in the leaflet and cannot be dissipated by a flexible support structure. Incorporation of this feature into percutaneous heart valves and especially balloon expandable percutaneous heart valves is much more challenging than in surgically implanted prostheses.

For a fully functioning prosthetic heart valve it is crucial that all of its components fulfill their respective task: The valve, on the one hand, needs to be adequately attached to the frame/stent support, since otherwise the valve is prone to failure, and valve failure, in the circulatory system, has significant consequences for the patient. On the other hand, the stent support needs to fully expand and, thus, guarantee the secure fixation within the heart vessels.

Further, the loading of the valve onto a deployment system for minimally invasive procedures can be quite challenging, since the heart valve prosthesis—due to its nature—needs to be carefully loaded while at the same time its compression is mandatory for getting it tightly packed onto the deployment system.

Thus, a need exists for an improved heart valve prosthesis device and methods of manufacturing the same.

### SUMMARY

According to the invention, this and other objects are solved by a Heart valve prosthesis for implantation into the heart of a patient, wherein the heart valve prosthesis comprises: a stent frame including a lumen, a luminal and abluminal side and a plurality of cells arranged in circumferential rows, wherein the plurality of cells comprises at least a proximalmost row of cells, at least one, preferably two, three, four or five, intermediate row of cells, and a distalmost row of cells; a valve element being mounted to the prosthetic heart valve and comprising at least two, preferably three, leaflets; a skirt attached to the stent frame, wherein the skirt comprises a skirt main body and at least two, preferably three, skirt flaps being integral with or being attached to the skirt main body, wherein the skirt main body covers at least the proximalmost row of cells, preferably on the luminal side, wherein each of the skirt flaps covers one respective commissure cell of the distalmost row of cells, thus forming a decoupling element, and wherein the leaflets, via the respective decoupling element, are mounted to the heart valve prosthesis within the commissure cells of the distalmost row of cells.

With the prosthetic heart valve of the invention, or rather due to the specific design, i.e. with the decoupling element of a specified flexibility provided in the heart valve prosthesis of the invention, commissures with a defined flexibility are generated, i.e. a flexible attachment of the valve via the commissures of the leaflets. Compared to the known heart valve prostheses, which have usually fixed/sewn the commissures to the stent frame, this design provides for a better, since more flexible function of the valve as a whole; further, due to the tailored flexibility of the commissures, the tensioning stress exerted on the valve as such is decreased when it is dissipated by the flexible decoupling element which overall leads to a prolonged lifetime of the prosthetic device/heart valve prosthesis.

Presently, with “decoupling element” an element having a defined flexibility is meant by means of which the fixation of the leaflet is decoupled from the stent frame or stent support, such, that it is not attached or fixed (e.g. sewn) directly to the stent frame or a part of the stent frame, thus uncoupling the mechanical load of the commissures and leaflets from the stent frame.

According to an embodiment of the invention, the decoupling element is formed by each of the skirt flaps in a folded state, i.e. a state where the skirt flap is bended, so that one part of the skirt flap lies on the other part.

Thus, the defined or specified flexibility of the decoupling element can be effected, e.g. by using a certain material for the skirt flap(s).

Accordingly, in an embodiment, the skirt flaps have a defined flexibility wherein the defined flexibility is defined by a Young's modulus/tensile modulus of between 50 to 3000 MPa, preferably between 100 to 500 MPa, tested with uniaxial tensile test machine according to ISO 527-1. With the defined flexibility, the mechanical loads experienced by the leaflets can be decoupled from the stent frame.

Herein, and as generally understood, the Young's modulus is understood as the modulus of elasticity in tension or compression (i.e., negative tension), and is a mechanical property that measures the tensile or compressive stiffness of a solid material when the force is applied lengthwise. The Young's modulus, thus, is indicative for a specified flexibility. The Young's modulus is a mechanical material property generally known in engineering design, and in material science/development in particular. E.g., one way of determining the Young's modulus/tensile modulus is applying a tensile test standard, wherein the Young's modulus is defined as the ratio of stress to strain during elastic loading, and can be determined according to ISO 527-1, or ISO 13934-1. In one embodiment, the determination of the Young's modulus can be performed, e.g., by the following method: the decoupling element is mounted in a uniaxial tensile testing machine and pulled to the end of the elastic limit, the yield strength. The resulting coefficient of proportionality between stress and strain gives the modulus of elasticity i.e. Young's modulus.

The defined flexibility can be effected, e.g., by using a certain material for the skirt flaps, as will be defined below. Also the material for the skirt flaps can be identical or different as the material for the skirt body. Also the skirt flaps may comprise an additional material to locally reinforce the decoupling element formed by the skirt flaps.

In an embodiment of the prosthetic heart valve of the invention, due to the skirt flaps having a defined flexibility, also the decoupling element has a specified flexibility, which can also be expressed by a Young's modulus that is similar/identical with the Young's modulus of the skirt flaps (in case where there are no reinforcements between the skirt flaps) and/or is preferably of between 50 to 3000 MPa, preferably of between 100 to 500 MPa, and more preferably between 150 and 300 MPa.

Via the de- or uncoupling element a mechanism is provided which allows a flexible attachment of the leaflets in the heart valve prosthesis device, which in turn leads to a better functioning of the valve as a whole: the flexible valve element, when closing, cannot dissipate mechanical loads into the rather stiff stent frame, but the decoupling element allows for this transfer of mechanical load into the stent frame. By reducing the level of stresses in the leaflets, the lifetime of the valve, thus, is prolonged.

Presently, and as generally understood, the meaning of terms referring to the anatomy of the heart have the same meaning as generally understood in the art, which is partly discussed at the outset of this invention.

Also, the term “skirt” as used in the present invention, and as generally understood in the field, means a sealing system, usually a thin layer/film made of biological or artificial material(s) attached to the stent frame to prevent paravalvular leaking. The skirt's attachment to the stent frame is usually effected by sewing or chemically attaching the skirt frame to the stent frame.

For definition purposes and according to the invention, a “stent” or “stent frame” refers to a structural component that is able to anchor in the tissue of an annular heart valve space. A stent frame is usually and preferably formed from a biocompatible metal frame, such as stainless steel or Nitinol, and can be, e.g., laser-cut or braided or otherwise made from interwoven wire filaments. Other stents that can be preferably used with the valves of the present invention include rigid rings, spiral-wound tubes, and other such tubes that fit tightly within an annular valve space and define an orifice through it for the passage of blood. The stent frame usually and preferably has a tubular or hollow-cylindrical shape.

Within the present invention, and as generally understood, a “valve” or “valve member/element” refers to that component of a heart valve that has fluid occlusion surfaces to prevent blood flow in one direction while allowing it in another as mentioned above, various constructions of valve members are available, including those with flexible leaflets and those with rigid leaflets, or even a ball and cage arrangement. The leaflets can be bioprosthetic, synthetic, metallic, or other suitable means.

As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. As used herein, the terms “substantial” and “substantially” or “essentially” mean, when comparing various parts to one another, that the parts being compared are equal to or are so close enough in dimension that one skill in the art would consider the same. Substantial and substantially and essentially, as used herein, are not limited to a single dimension and specifically include a range of values for those parts being compared. The range of values, both above and below (e.g., greater/lesser or larger/smaller), includes a variance that one skilled in the art would know to be a reasonable tolerance for the parts mentioned.

As used herein, the terms “integrally formed”, or “integral” and “unitary construction” refer to a construction that does not include any welds, fasteners, or other means for securing separately formed pieces of material to each other.

Also, as used herein, “directly attached to” when addressing the skirt main body and skirt flaps, means a direct connection of the skirt main body and the skirt flaps. E.g., a skirt body of a first material can be attached to the skirt flaps made from a different material. As such, “attached to” comprises means for securing separately formed pieces of the same or different material(s) to each other.

In the context of the present invention, the terms “proximal” and “distal” are used interchangeably with the terms “inflow” and “outflow”, respectively, so that, e.g., the “proximalmost” cells of the stent frame are the ones at the (blood) inflow end, and the distalmost cells are the cells at the (blood) outflow end of the stent frame. In other words, the proximalmost row of cells is the very last row of cells at the proximal end of the stent frame, and the distalmost row of cells is the very last row of cells at the distal end of the stent frame. As such, the stent frame comprises a proximal (inflow) end and a distal (outflow) end. Thus, for example, the heart valve prosthesis illustrated in FIG. 1 is shown in the orientation associated with implantation in the heart valve, and so the proximalmost row of the cells of the stent frame represent the ones at its inflow end and the distalmost row of the cells of the stent frame represent the one at the outflow end.

The term “cell”, as used herein, refers to a closed compartment in the stent frame, forming a “hole” in the stent frame (if uncovered by prosthetic material attached to the stent frame), which cell can be formed in different shapes by surrounding stent-material or stent struts. A row of cells represents a series of cells lying directly adjacent to one another seen in the circumferential direction of a tubular stent frame, wherein an adjacent cell shares a common edge or strut with the cell lying adjacent left or right to the cell.

A “skirt flap” within the meaning of the present invention is a piece of the skirt that extends from the skirt main body and which is attached to/is integral with the skirt main body with one side only and which extends freely with the other side.

According to a preferred embodiment of the invention, the decoupling element is effect, such, that the leaflets are mounted to the prosthetic heart valve via the decoupling element(s) within the commissure cells of the distalmost row of cells respectively covered by the skirt flaps. In that way, the skirt flaps covering the cells function as decoupling elements.

With this embodiment, advantageously, the skirt flaps covering the commissure cells of the distalmost row of cells are used as attachment zone; since the skirt flaps as such have a specified flexibility, also the leaflets retain their flexibility upon the attachment thereto.

Also, according to the invention, the leaflets do not contact the stent frame at the commissure level.

According to an embodiment of the heart valve prosthesis of the invention, each of the skirt flaps comprises an opening, such, that the opening is positioned within the commissure cell respectively covered by each of the skirt flaps.

This embodiment has the advantage that via the openings the leaflets can be attached to the skirt flaps, while maintaining the flexibility of the skirt flaps and the attachment thereto.

In an embodiment of the prosthetic heart valve of the invention, the skirt main body is attached on the luminal side to the stent frame.

In an embodiment of the prosthetic heart valve of the invention, the skirt main body is preferably attached on the luminal side of the stent frame, and preferably each of the skirt flaps comprises a first portion and a second portion, the first portion being attached on the luminal side of the stent frame and comprising an opening, which is positioned within said at least one commissure cell, and preferably, the second portion being designed for getting folded, from the luminal side of the stent frame to the abluminal side of the stent frame, over a distalmost edge of the commissure cell, such, that the second portion is abluminally attached to the stent frame, thereby at least partially covering the opening.

With this embodiment the skirt flaps are also used as attachment means for the leaflets: the leaflets, from the luminal side of the stent frame, can be guided through the opening in the skirt flap's first portion covering the cells, respectively, and the second portion can be folded over the distalmost edge of the commissure cell, thus at least partially covering the opening and the commissure cell the second portion is folded over from the luminal side to the abluminal side of the stent frame. Thus, an even more secure and flexible attachment of the leaflets can be achieved.

Thus, in an embodiment, the first portion of the skirt flaps is attached to the stent frame on the luminal side of the stent frame, and the second portion of the skirt flaps on the abluminal side of the stent frame.

In an another embodiment, each of the skirt flaps comprises a first portion and a second portion, the first portion being attached at the luminal side and comprising an opening, which opening is positioned within said at least one commissure cell, and preferably, the second portion being designed for getting folded, from the luminal side of the stent frame, around side struts of the commissure cell, such, that the second portion is abluminally attached to the stent frame, thereby at least partially covering the opening.

In another embodiment, the skirt main body is attached on the luminal side of the stent frame, each of the skirt flaps comprises a first portion and a second portion, wherein the first portion is attached to the abluminal side, and wherein the second portion is being designed for getting folded, from the abluminal side of the stent frame, around side struts of the commissure cell to the luminal side of the stent frame, such, that the second portion is luminally attached to the stent frame, such, that an opening is formed on the luminal side.

Accordingly, in an embodiment of the heart valve prosthesis of the invention, each of the leaflets comprises a leaflet main body and leaflet flaps, which leaflet main body is provided within the lumen of the stent frame, and which leaflet flaps are guided, through an opening positioned within said cell covered by a skirt flap, from the luminal to the abluminal side of the stent frame, for fixing each of the leaflets at/via the decoupling element(s).

In an embodiment of the heart valve prosthesis of the invention, the distalmost row of cells comprises at least two, preferably three, commissure cells, which are spaced apart from one another by separating cells, wherein the commissure cells are longer than the separating cells, thus protruding in the distal direction D.

With this embodiment, or rather with the specific commissure cells, the anatomy of the heart can be addressed. By the provision of only two or three longer commissure cells, the distalmost edge of the heart valve prosthesis does not circumferentially uniformly cover the site of the vessel the prosthesis is placed into; rather, via the shorter separating cells, space between the longer commissure cells is generated allowing to leave open de-branching arteries, such as, e.g., the coronary arteries.

Thus, in a preferred embodiment, the distalmost row of cells comprises three commissure cells, separated from one another by three separating cells, respectively, which commissure cells are longer than the separating cells.

Within the present invention, the term “longer” means that the commissure cells protrude in the distal direction about a certain length x as compared to the shorter separating cells. The length x may be from about 2 mm to about 8 mm, preferably smaller or equal to 5 mm.

Also, within the present invention, the term “commissure cells” designates the cells which serve as attachment zone of the leaflets of the valve element, thus generating the commissures of the valve element. Accordingly, the “separating cells” designate cells in the distalmost row that separate the commissure cells from one another.

In an embodiment of the present invention, the commissure cells have an essentially rhombus shape, and optionally, wherein the separating cells have an essentially deltoid shape.

This specific design of the commissure cells and the separating cells also provides for a design of the stent frame, by means of which the distal portion of the heart valve prosthesis allows a secure anchoring in the area of the native valve, while at the same time guaranteeing that the replacement valve element can fully function, while de-branching arteries are not blocked by the prosthesis.

In an embodiment of the heart valve prosthesis of the invention, the proximalmost row of cells comprises cells having an essentially heart shape.

With this design of the shape of the proximalmost row of cells, an overall better adjustment to the heart wall and better sealing can be achieved. Also, with the specific heart-shaped cells, the radial force is reduced which advantageously avoids a stimulus interruption.

In an embodiment of the heart valve prosthesis of the invention, the stent frame comprises at least one intermediate row lying directly adjacent to the distalmost row, which preferably comprises cells having an essentially deltoid shape.

With this deltoid-shaped cells, an asymmetric design of the cells is created allowing the cells to additionally keep de-branching arteries, such as the coronary arteries, open.

According to a preferred embodiment, the number of cells of the intermediate row lying directly adjacent to the distalmost row comprises between 6 and 16, cells, i.e. 6, 7, 8, 9, 10, 11, 12, 13, 14, 15 or 16 cells, preferably 12 cells.

In an embodiment of the heart valve prosthesis of the invention, the stent frame comprises a first intermediate row lying directly adjacent to the proximalmost row, which preferably comprises cells having an essentially hexagonal shape.

In an embodiment of the heart valve prosthesis of the invention, the stent frame comprises a second intermediate row lying directly adjacent to the first intermediate row, wherein preferably the second intermediate row comprises cells having an essentially hexagonal shape.

Preferably, the stent frame comprises a second intermediate row lying directly adjacent to the first intermediate row, which second intermediate row preferably comprises cells having an essentially hexagonal shape.

The first and the second intermediate row, in an embodiment of the invention, each comprise between 6 and 16 cells, preferably each between 10 and 12 cells, each more preferably 12 cells.

In an embodiment of the heart valve prosthesis of the invention, the first and second intermediate row comprises symmetric cells.

An embodiment of the heart valve prosthesis of the invention comprises a stent frame having 5 rows of cells, a distalmost row of cells, three intermediate rows of cells and a proximalmost row of cells, wherein the distalmost and proximalmost row of cells comprise cells having a shape as defined above, and wherein the three intermediate rows comprise cells having a shape as defined above for the intermediate row lying directly adjacent to the distalmost row (in the proximal direction) and for the first intermediate row lying directly adjacent to the proximalmost row (in the distal direction), and the second intermediate row lying adjacent to the first intermediate row (in the distal direction).

In an embodiment of the heart valve prosthesis of the invention, the skirt, i.e. the skirt flaps and/or the skirt main body comprises a fabric, film or tissue material of defined—and preferably uniform—flexibility, and preferably comprises a material selected from polyester, polyurethane, polytetrafluoroethylene (PTFE).

By providing a material that has a specified and uniform flexibility, the specified flexibility of the decoupling element as discussed above is guaranteed, and as a consequence, a secure attachment of the leaflets with defined flexibility is achieved.

The valve element, or rather the leaflets of the valve elements, are preferably made from pericardium of a mammal.

In an embodiment of the heart valve prosthesis of the invention the stent frame is balloon-expandable or self-expanding.

A self-expanding stent frame can be crimped or otherwise compressed into a small tube and has sufficient elasticity to expand on its own when a restriction such as an outer sheath is removed. In contrast, a balloon expansion stent frame is made of a material that is substantially less elastic, and must be plastically expanded from the inside to shape when converting from a contracted diameter to an expanded one. It should be understood that the term “balloon-expandable stents frames” includes plastically expandable stent frames, whether or not a balloon is used to actually expand them (for example, a device with mechanical fingers could expand the stent frame). The stent frame material deforms plastically after the application of a deformation force such as an inflation balloon or mechanical expansion fingers. Consequently, the term “balloon-expandable stent” should be understood as referring to the material or type of the stent frame as opposed to the specific expansion medium.

The present invention also concerns a method for manufacturing a heart valve prosthesis, the method comprising the steps of:
providing a stent frame including a lumen, a luminal and abluminal side and a plurality of cells arranged in circumferential rows, wherein the plurality of cells comprises at least a proximalmost row of cells, an intermediate row of cells, and a distalmost row of cells,
attaching, preferably stitching, a skirt to the stent frame, wherein the skirt comprises a skirt main body and at least two, preferably three, skirt flaps being integral with the skirt main body, such, that the skirt main body, at the luminal side of the stent frame, covers at least the proximalmost row of cells, and that each of the skirt flaps, with a respective first portion that comprises an opening, covers a cell in the distalmost row of cells at the luminal side of the stent frame, and with a second portion extending over the distalmost edge of said covered cell,
attaching at least two, preferably three, leaflets to the stent frame, wherein each of the leaflets comprises a leaflet main body and leaflet flaps, which leaflet main body is provided within the lumen of the stent frame, and which leaflet flaps are guided through the opening of the skirt flap's first portion from the luminal side to the abluminal side of the stent frame,
folding the respective second portion of the skirt flaps over the distalmost edge of said covered cells, so as to cover at least partially the leaflet flaps and the opening with the second portion,
attaching the second skirt flap portion to the stent frame, thereby fixating the leaflets to the stent frame via the decoupling element.

With the method as provided herein, a secure attachment of the valve element within the stent frame is guaranteed, while at the same time a flexible attachment of the valve is achieved, allowing an optimized stress distribution, and, as a consequence effecting the valve to operate and function as natural as possible.

In the heart valve prosthesis and the method of the invention, the expression “fixing/fixating the leaflets to the stent frame via the decoupling element” means that the leaflets are not directly attached to the stent frame, but rather directly to the decoupling element (and indirectly to the stent frame), which decoupling element in turn is integrated with/is connected with the stent frame.

In an embodiment of the method of the invention, each leaflet comprises two flaps, which are guided through the opening in a folded state, and which are unfolded after the passage through the opening, and subsequently covered by the skirt flap's second portion.

Also, in an embodiment of the method of the invention, a heart valve prosthesis as disclosed above is manufactured.

The heart valve prosthesis/prosthetic device of the invention is suitable/used for replacing any of the valves of a heart of a mammalian patient, preferably a human, preferably an adult human heart, i.e. the aortic valve, the pulmonary valve, the mitral valve, and the tricuspid valve, wherein a replacement of the aortic valve is preferred.

Accordingly, in the present invention also concerns a method for treating a defective native valve in a mammalian, preferably human, heart to replace the function of the native valve, wherein the native valve preferably is an aortic valve, wherein the method for treating comprises the step of: —advancing a heart valve prosthesis as disclosed above through the vasculature of a patient to a treatment site along a wall of a blood vessel; —either (i) expanding an expansion member within the stent frame such that the stent frame expands and contacts the vessel wall, or (ii) withdrawing a sheath holding compressed the heart valve prosthesis such that the stent frame expands and contacts the vessel wall upon withdrawing the sheath.

It is understood that the features described hereinabove and those still to be described below fall within the scope of the present invention not only in the respectively specified combinations, but also in different combinations or on their own, such, that the disclosure should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g., each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

### BRIEF DESCRIPTION OF THE DRAWINGS

Preferred embodiments are shown in the figures and are described in further detail herein below.

In the figures:

FIG. 1A shows a schematic drawing of pattern of a stent frame of an embodiment of the heart valve prosthesis of the invention, with the otherwise tubular form flattened out, without the outlines of a valve; FIG. 1B shows this stent frame with the schematic outlines of an attached valve; FIG. 1C shows a drawing of the stent frame in tubular form;

FIG. 2 shows a schematic drawing of a skirt element used in an embodiment of the heart valve prosthesis of the invention, flattened out and unattached to the stent frame;

FIG. 3A shows a schematic drawing of a singular leaflet of a valve element of an embodiment of the heart valve prosthesis of the invention in a first shape; FIG. 3B shows a schematic drawing of a singular leaflet of a valve element in a second, different shape;

FIG. 4A shows an enlarged view of a portion of the stent frame and shows a first part/step of the method for manufacturing a heart valve prosthesis of the invention, with the skirt getting/being attached to the stent frame of an embodiment of the heart valve prosthesis of the invention, with the skirt main body covering the proximalmost and intermediate rows of the stent frame, wherein a first portion of the skirt flap covers a cell of the distalmost row of cells of the stent frame, without being folded over and without the leaflet being guided through the opening; FIG. 4B shows another, second step of the method for manufacturing a heart valve prosthesis of the invention, with the leaflet being partially guided through the opening and without the second portion of the skirt flap being folded over; and FIG. 4C shows yet another, third step of the method for manufacturing a heart valve prosthesis of the invention, with the second portion of the skirt flap being folded over the edge of the cell covered by the first portion;

FIG. 5A shows a schematic drawing of a cross-section of a part of the stent frame of an embodiment of the heart valve prosthesis of the invention, with leaflet flaps being partially guided through the opening in the first portion of the skirt flap covering a cell in the distalmost row of the stent frame; in the embodiment shown in FIG. 5A, the second portion of the skirt flap is folded over a distalmost edge of the commissure cell without being wrapped around the struts of the stent frame's commissure cell; in the embodiment shown in FIG. 5B, the second portion of the skirt flap is folded over a distalmost edge of the commissure cell, with the flap being wrapped around the struts of the stent frame's commissure cell; and in the embodiment shown in FIG. 5C, the second portion of the skirt flap being folded over a distalmost edge of the commissure cell, with the flap being wrapped around the struts of the stent frame's commissure cell and with the second portion of the flap being divided and overlapping each other.

FIG. 6A shows a schematic drawing of the mechanism of the decoupling element in a front view; and FIG. 6B shows a cross-section through the stent frame; and

FIG. 7 shows a drawing of an embodiment of the assembled heart valve prosthesis of the invention in a perspective view.

### DETAILED DESCRIPTION OF EMBODIMENTS OF THE INVENTION

The accompanying drawings, which are incorporated herein and form a part of the specification, further serve to explain the principles of the invention and to enable a person skilled in the pertinent art to make and use the invention. The drawings are not to scale.

Specific embodiments of the present invention are now described with reference to the figures, wherein like reference numbers indicate identical or functionally similar elements. The following detailed description is merely exemplary in nature and is not intended to limit the invention and uses of the invention. Furthermore, there is no intention to be bound by any expressed or implied theory presented in the preceding technical field, background, brief summary or the following detailed description.

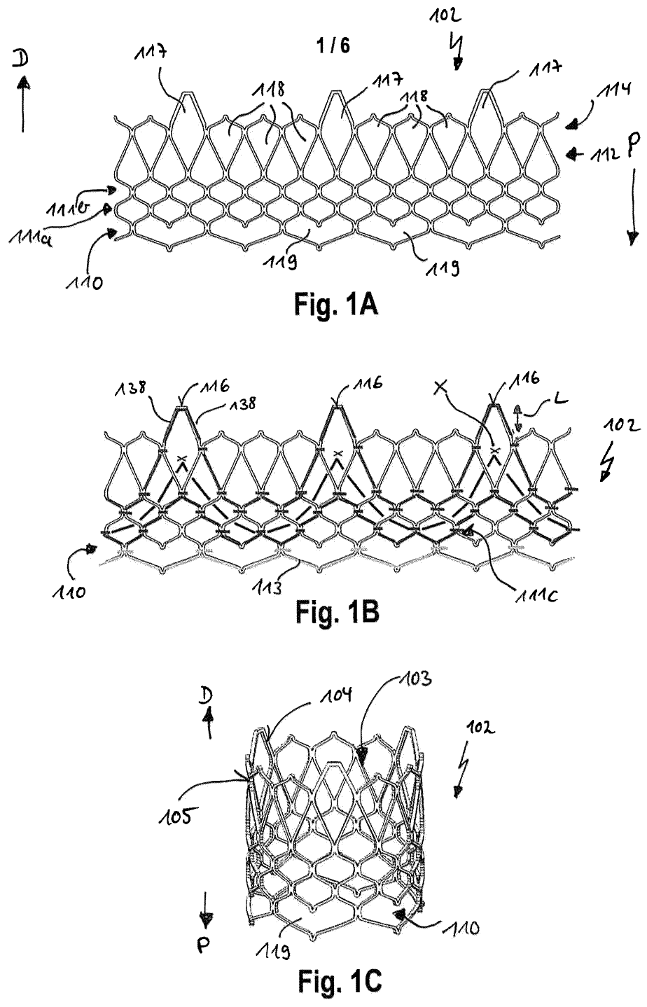

FIG. 1, in 1A and 1B, shows a schematic drawing of pattern of a stent frame 102 of an embodiment of a heart valve prosthesis 100 of the invention, with the otherwise tubular closed form flattened out/opened. As can be seen in FIG. 1A, the stent frame 102 comprises a plurality of circumferential rows 110, 111a, 111b, 112, 114, with a proximalmost row 110, a distalmost row 114, an intermediate row 112 lying directly adjacent to the distalmost row 114 (in the proximal direction P), a first intermediate row 111a lying directly adjacent to the proximalmost row 110 (in the distal direction D) and a second intermediate row 111b lying directly adjacent to the first intermediate row 111a.

As can be seen in FIGS. 1A, 1B and 1C, the proximalmost row 110 comprises cells, which have an essentially heart shape. As a consequence of the heart shape, the cells in the proximalmost row 110, in the embodiment shown here, are larger/have a larger opening than the first intermediate row 111a and the second intermediate row 111b.

The heart shape is exemplary only, other shapes effecting the same purpose, i.e. a better attachment and sealing to the heart wall, can be employed, too.

The cells of the first and the second intermediate rows 111a and 111b have an essentially hexagonal shape, and are, in the embodiment shown in FIG. 1, smaller than the cells of the proximalmost row of cells 110. As a consequence, also the number of cells contained in the first and second rows 111a and 111b are higher. In the embodiment shown in FIG. 1, in particular in FIG. 1 C, the number of cells in the proximalmost row is 6, and the numbers of cells in the first and second intermediate rows 111a and 111b is 12, respectively. However, it is to be understood, that the number of cells is only exemplary; other numbers/ranges are feasible to effect the same purpose.

In the embodiment depicted in FIG. 1, the distalmost row of cells 114 comprises cells, wherein three larger cells, also designated as commissure cells 117, are, respectively, spaced apart from one another by separating cells 118. In the embodiment shown in FIG. 1, the three commissure cells 117 are respectively separated from one another by three separating cells 118. The commissure cells 117 are larger/longer than the separating cells 118, thus protruding in the distal direction D as compared to the shorter separating cells 118. The length L the commissure cells are longer than the shorter separating cells, or, in other words, are protruding from the shorter separating cells, is from about 2 mm to about 2 cm, preferably from 2 mm to 8 mm.

The three commissure cells 117 represent the cells 117, where the commissures are mounted (via the skirt, see description below). The two rows 111b and 111a effect a radial force for fixation of the heart valve prosthesis in the implantation site.

The proximalmost row of cells 110 comprises, in the embodiment shown in FIG. 1, 6 heart-shaped cells, thus providing for a lower radial force and for a higher flexibility in order to effect a better adaptation to the annulus of the native heart valve to be replaced. The different shape of the row of cells effects a different gradient of struts interconnecting the cells. By having a different gradient, the radial force is decreased to avoid stimulus interruption.

Also schematically depicted in FIG. 1B is the attachment of the commissures (via the skirt 130; not shown in FIG. 1; but see description below), indicated with three crosses x, as well as of the outline of the valve leaflets VL/semilunar junction: The top point x is inside the commissure cell 117, and the lowest point is 111c between two cells in the first intermediate cell row 111a above the proximalmost or inflow row 110.

The commissure cells 117 of the distalmost row of cells 114 of the heart valve prosthesis 100 shown in FIG. 1 have an essentially rhombus shape, and the separating cells 118 have an essentially deltoid shape.

In FIG. 1C, the stent frame 102 is shown in its tubular shape, thus having a lumen 103, a luminal side 104 and an abluminal side 105. As can be seen also in this figure, the stent frame 102 of this embodiment comprises 5 rows of cells, the proximalmost row representing the inflow end, and the distalmost row 114 representing the outflow end of the stent frame 102.

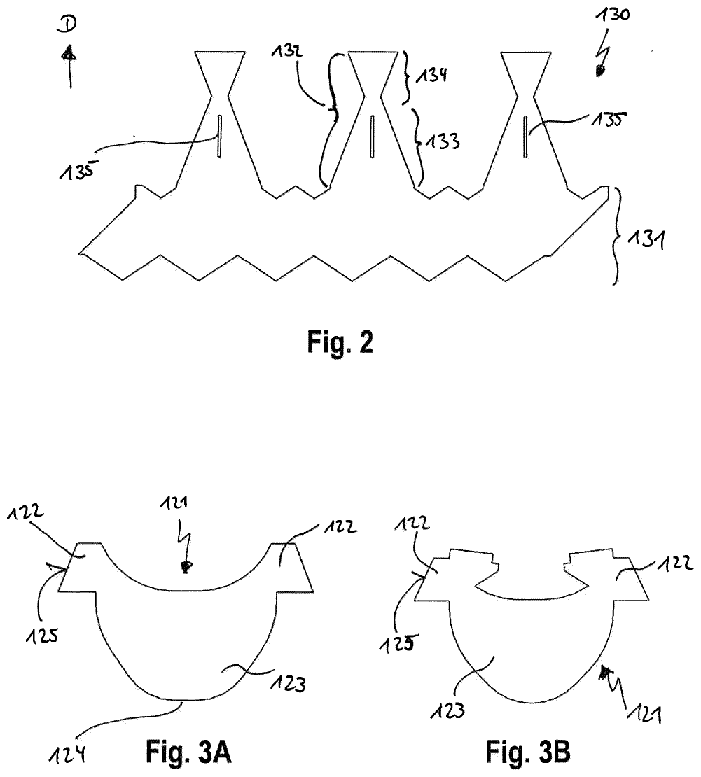

FIG. 2 shows a schematic drawing of a skirt 130, which, in the assembled state of the heart valve prosthesis 100, is—with its main body 131 and first portions 133 of skirt flaps 132—attached to/provided on the luminal side 104 of the stent frame 102. As for the stent frame 102 in FIGS. 1A and 1B, the skirt 130 in FIG. 2, for better understanding, is not shown attached to the stent frame 102 and is also not shown in the tubular form, but in open/flattened out form.

As can be seen from FIG. 2, the skirt comprises a skirt main body 131 and three skirt flaps 132 integrally formed with and extending from the skirt main body 131 in distal direction D. The three skirt flaps 132, in the assembled state of the heart valve prosthesis of the invention, cover the three commissure cells 117 of the distalmost row 114 of stent frame 102.

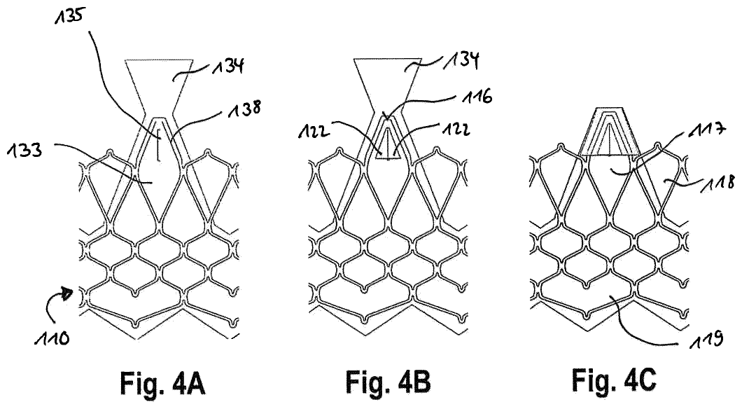

Each of the skirt flaps 132 comprises a first portion 133 and a second portion 134, respectively. The respective first portions 133 comprise openings 135 (or windows or holes), which, in the assembled state of the heart valve prosthesis of the invention, i.e. with the skirt 130 being attached the stent frame 102, are positioned within cells 117, i.e. commissure cells. For assembling the heart valve prosthesis of the invention, the skirt 130 is attached on the luminal side 104 of the stent frame 102, with the skirt main body 131 covering at least the proximalmost row of cells 110 from the inside of lumen 103. The respective second portions 134 of the skirt flaps 132 are designed such, that they, from the luminal side 104, can be folded over a distalmost edge 116 of the commissure cell 117 (see FIG. 1), such, that the second portion 134 is abluminally attached to the stent frame 102. The assembly of the skirt 130 to the stent frame is shown in more detail in FIG. 4, which is described further below.

The schematic drawing of the skirt 130 as shown in FIG. 2 shows that the proximalmost outline 136 or shape of the skirt 130 adapts to the outline of the proximalmost outline 113 of the stent frame 102.

In the assembled state of the heart valve prosthesis 100 of the invention, thus, the skirt is attached to the complete height of the stent frame 102.

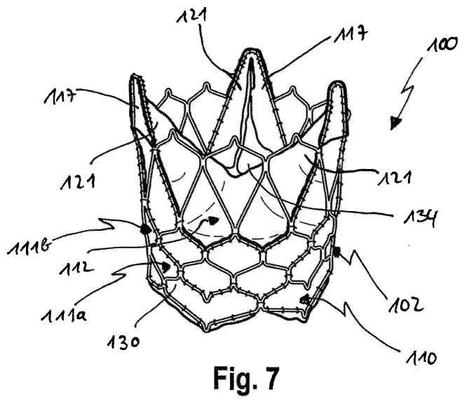

FIG. 3 depicts schematic drawings of leaflets 121 of a valve element 120 (see FIG. 7) of an embodiment of the heart valve prosthesis 100 of the invention, exemplarily in two different shapes (A) and (B) of a singular leaflet 121, not attached to the stent frame 102.

As shown in FIG. 3, each leaflet 121 comprises a leaflet main body 123 and two leaflet flaps 122 or wings. The leaflet main body 123 is provided within the lumen 103 of the stent frame 102, and the leaflet flaps 122 are guided, through openings 135 in the skirt flaps 132 and positioned within the respective commissure cells 117 covered by the skirt flaps 132, from the luminal side 104 to the abluminal side 105 of the stent frame 102, for fixing each of the leaflets 121 at the stent frame 102 via the decoupling element 99. The leaflet flaps 122 are formed and designed such, that they can be guided through opening 135 of skirt flap 132 as will be described below for FIG. 4.

As can be seen in FIG. 3A, 3B, the shape of the leaflet flaps 122 have a round free edge which is lower than the flaps 122. The leaflet flaps 122 have an oblique edge 125 corresponding to the cell of the stent frame.

FIG. 4 shows an enlarged view of a portion of the stent frame 102 and shows a part of the steps of the method for manufacturing a heart valve prosthesis 100 according to an embodiment of the invention. FIG. 4a shows the alignment of the skirt 130 in the luminal side 104 of the stent frame, such that the skirt main body 131 covers the proximalmost row 110, as well as the first intermediate row 111a and the second intermediate row 111b inside lumen 103. As can be seen in FIG. 4, the first portion 133 of a respective skirt flap 132 covers a commissure cell 117 of the distalmost row 114 of the stent frame 102, such, that opening 135 is positioned in the commissure cell 117. The second portion 134 of the skirt flap 132 is, in this step, extending beyond, in distal direction D, the proximalmost edge 116 of commissure cell 117. In other words, the second portion 134 of skirt flap 132 is not yet folded over the proximalmost edge 116 of the commissure cell. Also, the leaflet 121, or rather the leaflet flaps 122 or wings are not yet guided through opening/hole 135 positioned within commissure cell 117.

In a next step, shown in FIG. 4B the leaflets 121, or rather the leaflet flaps 122 are, from the luminal side 105 of the stent frame 102, guided or pushed through openings 135 positioned within the commissure cell 117, and folded to the right and left side of the opening 135, respectively, and thus, guided to the abluminal side 105 of the stent frame 102.

FIG. 4C shows that in a subsequent step the second portion 134 of the skirt flap 132 is folded over the proximalmost edge 115 of commissure cell 117, such, that it—at least partially—covers the opening 135 and the leaflet flaps 122. Subsequently, the second portion, in this position, can be attached, e.g. sewn to the stent frame 102 on its abluminal side 105, thus fixating the leaflets within the prosthetic heart valve. By this unique fixation modus, the leaflets do not contact the stent frame 102.

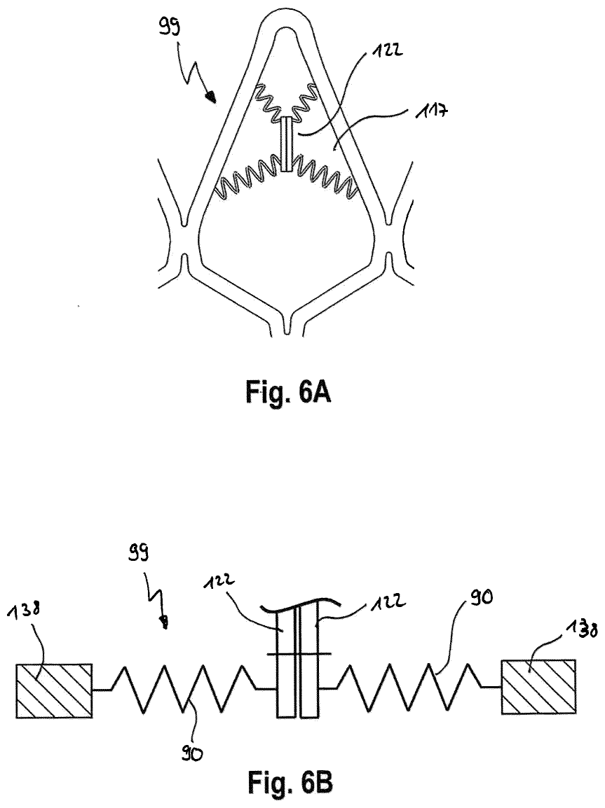

By the attachment method for the leaflet shown in FIG. 4, and as claimed therein, the leaflet is decoupled from the stent frame, providing for an improved flexibility of the valve element. Thus, the combination of the skirt 130, with the first portion 133 of the skirt flap 132 covering the commissure cell 117 and comprising the opening 115, and the second portion 134 being folded over the leaflet flaps 122 guided through the opening 115 provides for decoupling element 99 (see FIG. 6).

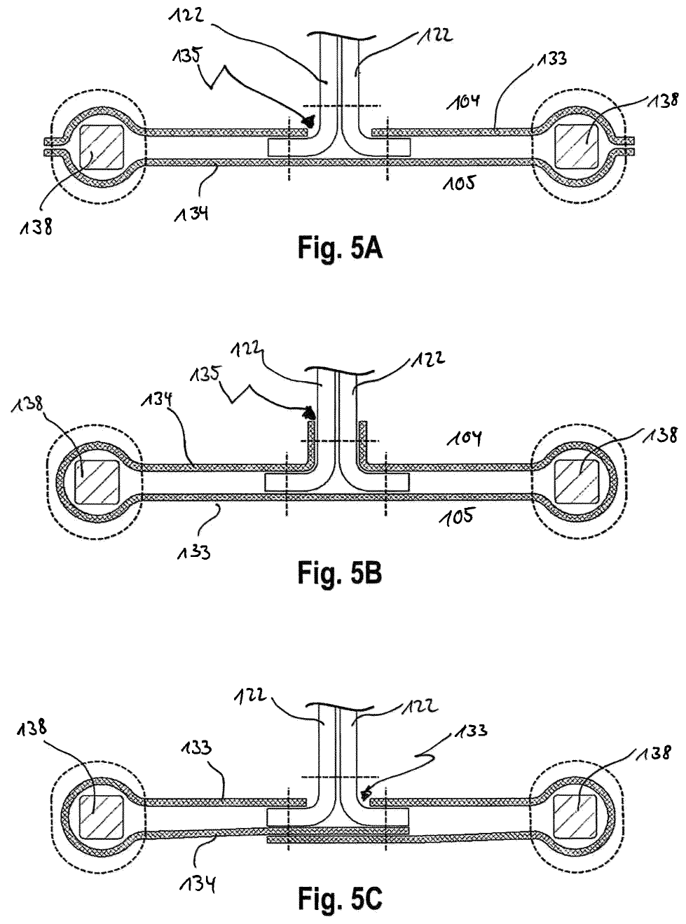

In FIG. 5, different embodiments for forming a decoupling element 99 by means of the skirt flaps 132 in relation to the stent frame 102, and for attaching the skirt/skirt flaps to the stent frame, are shown.

As such, FIGS. 5A to C each show a schematic drawing of a cross-section of a portion of the stent frame 102 of an embodiment of the heart valve prosthesis 100 of the invention is shown with the leaflet 121/leaflet flaps 122, being partially guided through opening 135 of the skirt flap 132 covering the commissure cell 117 in the distalmost row 114 of the stent frame 102. In FIG. 5, 138 designates the struts of the distalmost commissure cell in cross-section.

FIG. 5A shows an embodiment, where the first portion 133 of skirt flap 132 is luminally covering the stent frame 102, and the second portion 134 of the skirt flap 132 is abluminally covering the stent frame 102. The skirt flap 132 is not guided or wrapped around the commissure cell's struts 138. In this embodiment, also the skirt main body 131 is attached to the luminal side 104 of the stent frame 102.

FIG. 5B shows another embodiment for the attachment of the skirt element and for forming the decoupling element 99. Here, the first portion 133 of the skirt flap 132 (and as a consequence: also the main body 131 of the skirt 130, which, however is not shown in FIG. 5) is attached to the abluminal side 105 of the stent frame 102, and the second portion 134 of the skirt flap 132 is wrapped around the commissure cell's struts 138. On the luminal side 104 of the stent frame 102, an opening 135 is formed, e.g. by not joining the two ends of the skirt flap's 132 second portion 134 on the luminal side 104.

FIG. 5C shows yet another embodiment for the attachment of the skirt element and for forming the decoupling element 99. Here, the skirt main body 131 is attached from the luminal side 104 of the stent frame, and the first portion 133 of the skirt flap 132 is attached to the luminal side 104, too, and comprises an opening 135. The opening 135 is positioned within commissure cell 117. The second portion 134 of the skirt flap 132 is wrapped around the commissure cell's struts 138, such, that the second portion is abluminally attached to the stent frame 102, and abluminally covers opening 135. The ends 134a and 135b of the second portion can be layered one over the other, as depicted in FIG. 5C.

In FIG. 6, a schematic drawing of the mechanism of decoupling element 99 is shown, with a front view (A) and a cross-section through the stent frame 102 (B). As schematically depicted, the decoupling element 99 (see description above) provides for a flexible “suspension” for the leaflets in the heart valve prosthesis 100 of the invention, indicated by representative spring elements 90. The decoupling element, in the embodiment shown in FIG. 4, is effected by the leaflet's attachment via the skirt flaps 132 covering the commissure cells 117. Thus, in this embodiment, the skirt (or rather skirt portion) covering the commissure cell 117 effects the spring element's suspension for the commissure. In that way, as discussed above, the leaflets are decoupled from the stent frame 102 and the stent frame's 102 movement, effecting a flexible commissure, and, thus, a higher flexibility of the valve element as compared to an attachment to the stent frame/its struts directly.

FIG. 7 shows a drawing of an assembled embodiment of the heart valve of the invention. In FIG. 7, the valve element 120 can be seen, which, in the embodiment shown in FIG. 7 has three leaflets 121. The valve element 120 is attached within the lumen 103 of the stent frame 102. The skirt 130, with the skirt main body 131, is attached within the lumen 103 of the stent frame 120, luminally covering the proximalmost row 110 and the first and second intermediate row 111a and 111b. Separating cells 118 of the distalmost row of cells 114 are not covered, while the commissure cells 117 of the distalmost row 114 are covered, from the lumen side 103, by the first portion 133 of the skirt flap 132, and from the outside by the second skirt portion 134, which is folded over the distalmost edge of the commissure cells 117. The skirt is sewn to the stent frame using stiches to attach it to the struts forming the cells of the stent frame. With the uncovered cells in the distalmost row of cells 114 and the intermediate row 112 lying directly adjacent to the distalmost row 114, there is provided enough space for outwash.

What is claimed is:

1. Heart valve prosthesis for implantation into the heart of a patient, wherein the heart valve prosthesis comprises:
a stent frame including a lumen, a luminal and abluminal side and a plurality of cells arranged in circumferential rows, wherein the plurality of cells comprises at least a proximalmost row of cells, at least one intermediate row of cells, and a distalmost row of cells,
a valve element being mounted to the prosthetic heart valve and comprising at least two, preferably three, leaflets,
a skirt attached to the stent frame, wherein the skirt comprises a skirt main body and at least two, preferably three, skirt flaps being integral with or being attached to the skirt main body, wherein the skirt main body covers at least the proximalmost row of cells, and wherein each of the skirt flaps covers one respective commissure cell of the distalmost row of cells, and thus forming a decoupling element, and
wherein the leaflets, via the respective decoupling element, are mounted to the heart valve prosthesis within the commissure cells of the distalmost row of cells.

2. The heart valve prosthesis of claim 1, wherein each of the skirt flaps comprises an opening, such, that the opening is positioned within said commissure cell respectively covered by each of the skirt flaps.

3. The heart valve prosthesis of claim 1, wherein each of the skirt flaps comprises a first portion and a second portion, the first portion comprising an opening, which is positioned within said at least one commissure cell, and the second portion being designed for getting folded over a distalmost edge of the commissure cell, such, that the second portion is abluminally attached to the stent frame, thereby at least partially covering the opening.

4. The heart valve prosthesis of claim 1, wherein the skirt main body is attached on the luminal side of the stent frame, each of the skirt flaps comprises a first portion and a second portion, wherein the first portion is attached to the abluminal side, and wherein the second portion is designed for getting folded, from the abluminal side of the stent frame, around side struts of the commissure cell, such, that the second portion is luminally attached to the stent frame, such, that an opening is formed on the luminal side.

5. The heart valve prosthesis of claim 1, wherein each of the skirt flaps comprises a first portion and a second portion, the first portion being attached at the luminal side and comprising an opening, which opening is positioned within said at least one commissure cell, and the second portion is designed for getting folded, from the luminal side of the stent frame, around side struts of the commissure cell, such, that the second portion is abluminally attached to the stent frame, thereby at least partially covering the opening.

6. The heart valve prosthesis of claim 1, wherein each of the leaflets comprises a leaflet main body and leaflet flaps, which leaflet main body is provided within the lumen of the stent frame, and which leaflet flaps are guided, through an opening positioned within said commissure cell covered by a skirt flap, from the luminal to the abluminal side of the stent frame, for fixing each of the leaflets at the stent frame via the decoupling element.

7. The heart valve prosthesis of claim 1, wherein the distalmost row of cells comprises at least two, preferably three, commissure cells, which are spaced apart from one another by separating cells, wherein the commissure cells are longer than the separating cells, thus protruding in the distal direction D.

8. The heart valve prosthesis of claim 7, wherein the commissure cells have an essentially rhombus shape, and optionally, wherein the separating cells have an essentially deltoid shape.

9. The heart valve prosthesis of claim 1, wherein the proximalmost row of cells comprises cells having an essentially heart shape.

10. The heart valve prosthesis of claim 1, wherein the stent frame comprises an intermediate row lying directly adjacent to the distalmost row, preferably which comprises cells having an essentially deltoid shape.

11. The heart valve prosthesis of claim 1, wherein the stent frame comprises a first intermediate row lying directly adjacent to the proximalmost row, preferably which comprises cells having an essentially hexagonal shape, and preferably wherein the stent frame comprises a second intermediate row lying directly adjacent to the first intermediate row, wherein preferably the second intermediate row comprises cells having an essentially hexagonal shape.

12. The heart valve prosthesis of claim 1, wherein the skirt comprises a fabric, film or tissue material of defined flexibility, and preferably comprises a material selected from polyester, polyurethane, PTFE.

13. The heart valve prosthesis of claim 1, wherein the skirt main body of a skirt is provided on the luminal side of the stent frame, a respective first portion of the skirt flap is provided on the luminal side of the stent frame, and the second portion of the skirt flap is provided at the abluminal side of the stent frame.

14. The heart valve prosthesis of claim 1, wherein the stent frame is balloon-expandable or self-expanding.

15. A method for manufacturing a heart valve prosthesis, the method comprising the steps of:
providing a stent frame including a lumen, a luminal and abluminal side and a plurality of cells arranged in circumferential rows, wherein the plurality of cells comprises at least a proximalmost row of cells, an intermediate row of cells, and a distalmost row of cells,
attaching, preferably stitching, a skirt to the stent frame, wherein the skirt comprises a skirt main body and at least two, preferably three, skirt flaps being integral with the skirt main body, such, that the skirt main body, at the luminal side of the stent frame, covers at least the proximalmost row of cells, and that each of the skirt flaps, with a respective first portion that comprises an opening, covers a cell in the distalmost row of cells at the luminal side of the stent frame, and with a second portion extending over the distalmost edge of said covered cell,
attaching at least two, preferably three, leaflets to the stent frame, wherein each of the leaflets comprises a leaflet main body and leaflet flaps, which leaflet main body is provided within the lumen of the stent frame, and which leaflet flaps are guided through the opening of the skirt flap's first portion from the luminal side to the abluminal side of the stent frame,
folding the respective second portion of the skirt flaps over the distalmost edge of said covered cells, so as to cover at least partially the leaflet flaps and the opening with the second portion,
attaching the second skirt flap portion to the stent frame, thereby fixating the leaflets to the stent frame via the decoupling element.

16. The method of claim 15, wherein each leaflet comprises two flaps, which are guided through the opening in a folded state, and which are unfolded after the passage through the opening, and subsequently covered by the skirt flap's second portion.

17. The method of claim 15, wherein a heart valve prosthesis is manufactured, that comprises:
a stent frame including a lumen, a luminal and abluminal side and a plurality of cells arranged in circumferential rows, wherein the plurality of cells comprises at least a proximalmost row of cells, at least one intermediate row of cells, and a distalmost row of cells,
a valve element being mounted to the prosthetic heart valve and comprising at least two, preferably three, leaflets,
a skirt attached to the stent frame, wherein the skirt comprises a skirt main body and at least two, preferably three, skirt flaps being integral with or being attached to the skirt main body, wherein the skirt main body covers at least the proximalmost row of cells, and wherein each of the skirt flaps covers one respective commissure cell of the distalmost row of cells, and thus forming a decoupling element, and
wherein the leaflets, via the respective decoupling element, are mounted to the heart valve prosthesis within the commissure cells of the distalmost row of cells.
